(19) United States
(12) Patent Application Publication
(10) Pub. No.: US 2003/0228402 A1
(43) Pub. Date: Dec. 11, 2003

(54) Compositions and methods for preservation of food

(76) Inventors: Lanny U. Franklin, Atlanta, GA (US); Julio L. Pimentel, Buford, GA (US)

Correspondence Address: NEEDLE & ROSENBERG, P.C., SUITE 1000, 999 PEACHTREE STREET, ATLANTA, GA, 30309-3915, US

(21) Appl. No.: 10/368,779

(22) Filed: Feb. 19, 2003

Related U.S. Application Data
(60) Provisional application No. 60/358,088, filed on Feb. 19, 2002.

(51) Int. Cl. A23B 4/00; A23D 7/00; A61K 35/78

(52) U.S. Cl. 426/541; 424/439; 424/742

(57) ABSTRACT

Composition and methods for preservation of food and/or prevention of gastrointestinal infection. A composition comprising a single terpene, a terpene mixture, or a liposome-terpene(s) composition is disclosed. The composition can be a true solution of an effective amount of an effective terpene and a carrier such as water. The composition can be a suspension or emulsion of terpene, surfactant, and carrier. The composition(s) of the invention can be applied directly to food or to articles or devices used in food handling. Application can be, for example, by spraying a solution of the present invention. A true solution of terpene and water can be formed by mixing terpene and water at a solution-forming shear rate in the absence of a surfactant.

### CROSS REFERENCE TO RELATED APPLICATIONS

This application claims the benefit of U.S. Provisional Application 60/358,088, filed Feb. 19, 2002, hereby incorporated by reference.

### BACKGROUND OF THE INVENTION

1. Field of the Invention

A composition and method for preservation of all types of food and beverage.

2. Background

Because food is so important to survival, food preservation is one of the oldest technologies used by human beings. Unless sterilized and sealed, all food contains bacteria. Over the ages, various methods have been used, e.g., salting, pickling, drying/dehydration, canning, pasteurizing, fermentation, refrigeration, freezing, chemicals, and irradiation.

The basic idea behind all forms of food preservation is

(1) to slow the activity of disease-causing bacteria or

(2) to kill the bacteria.

In certain cases, a preservation technique may also destroy enzymes naturally found in a food that cause it to spoil or discolor quickly. By increasing the temperature of food to about 150° F. (66° C.), enzymes are destroyed.

Refrigeration and freezing are probably the most popular forms of food preservation in use today, at least in most developed areas of the world. The premise of refrigeration is to slow bacterial action so that it takes food much longer to spoil. Freezing is intended to stop bacterial action altogether. Refrigeration and freezing are used on almost all foods, e.g., meats, fruits, vegetables, beverages. In general, refrigeration has little to no effect on a food's taste or texture. Freezing's effect on the taste or texture depends on the food frozen, especially on the food's water content.

Since the 19^{th} century, canning has been a way for people to store foods for extremely long periods of time. In canning, the food is boiled in the can (or any sealable container) to kill all the bacteria and seal the can (either before or while the food is boiling) to prevent any new bacteria from getting in. Since the food in the can is completely sterile, it does not spoil. Once you open the can, bacteria enter and begin growing. One problem with canning is that the act of boiling food in the can generally changes its taste, texture, and nutritional content.

Many foods are dehydrated to preserve them. Examples of dehydrated products are powdered milk, dehydrated (“instant”) potatoes, dried fruits and vegetables, dried meats (e.g., beefjerky), powdered soups and sauces, pasta, and instant rice. Since most bacteria die or become inactive when dried, dried foods kept in air-tight containers can last a long time. Normally, drying completely alters the taste and texture of the food. Freeze drying is a particular form of drying involving freezing the food and placing it in a strong vacuum until the water sublimates. Freeze drying tends to have less of an effect on a food's taste than normal dehydration. Freeze-drying is commonly used to make instant coffee, but also works extremely well on other foods.

Salting, especially of meat, is an ancient preservation technique. The salt draws out moisture and creates an enviromnent inhospitable to bacteria. If salted in a cold environment (so that the food does not spoil while the salt has time to take effect), salted food, like meat, can last for years. Salting was used to preserve meat up through the middle of this century. Today, salting is still used to create salt-cured country ham, dried beef, and corned beef and pastrami.

Pickling was widely used to preserve meats, fruits, and vegetables in the past, but today is used almost exclusively to produce pickled cucumbers. Pickling uses the preservative. qualities of salt combined with the preservative qualities of acid, such as acetic acid (vinegar). Acidic environments inhibit bacteria.

Pasteurization is a compromise technique. Boiling food can kill all bacteria and make the food sterile, but it often significantly affects the taste and nutritional value of the food. Pasteurization involves heating food to a high enough temperature to kill certain (but not all) bacteria and to disable certain enzymes while minimizing the effects on taste. Commonly pasteurized foods include milk, ice cream, fruit juices, beer, and non-carbonated beverages. Ultra high temperature (UHT) pasteurization completely sterilizes the product. It is used to created boxes of milk.

Fermentation uses yeast to produce alcohol. Alcohol is a good preservative because it kills bacteria. Wine will last quite a long time (decades if necessary) without refrigeration. Normal grape juice would mold in days.

Chemical preservatives are also used to preserve foods. There are three classes of chemical preservatives commonly used in foods: benzoates (e.g., sodium benzoate), nitrites (e.g., sodium nitrite), and sulfites (e.g., sulfur dioxide). Another common preservative is sorbic acid. All of these chemicals either inhibit the activity of bacteria or kill the bacteria.

Radiation is able to kill bacteria without significantly changing the food which is treated. If food is sealed in a container and then irradiated, the food will become sterile and can be stored without refrigeration. Unlike canning, irradiation does not significantly change the taste or texture of the food. Irradiation of meats could prevent many forms of food poisoning.

One of the obvious objectives of food preservation is to avoid gastrointestinal (GI) infections.

Digestive tract infections are mainly caused by pathogenic and opportunistic microorganisms and their toxins. These infections are the product of inefficient microbial control during processing and packaging of food. Slaughter and processing houses are sources of contaminated meat and produce. For example, that is the case with the presence of E. coli in hamburger and Salmonella in eggs and poultry.

Salmonella and E. coli are becoming more common, but recently there has also been an increase of the incidence of Listeria, Clostridia, and other common microorganism in animals. Slaughter houses and processing plants use antimicrobials to clean and disinfect the plant, but the effectiveness is related to the amount of organic matter present during slaughtering, scalding, and chilling, and the cleanliness of the food packaging. Another factor involved in the greater incidence of digestive infections due to consumption of contaminated food is the lack of knowledge by the consumer that food in an optimum media for microbial growth and that precautions must be taken in order to avoid contamination. Contributing to issues of infection is the significant use of antibiotics in raising livestock which has in turn produced antibiotic-resistant strains of infectious agents.

Some of the symptoms of digestive infection include nausea, abdominal pain, and diarrhea. Diarrhea may be presented either as (1) acute watery diarrhea, (2) diarrhea with blood (dysentery), or (3) chronic diarrhea, often with clinical nutrient malabsorption. The common pathogens responsible include Clostridium difficile, Yersenia enterolitica, Shigella sp., Campylobacter sp., Salmonella sp., ETEC (enterotoxigenic) and EAEC (enteroaggregative) Escherichia coli. Viruses such as rotavirus, cytomegalovirus, and Norwalk agent are less common causes.

The use of antibiotics limits the course of diarrhea. The widespread resistance of the traditional antimicrobial agents, trimethoprim plus sulfamethoxazole (TMP/SMX) and fluoroquinolones, are the main reasons of concern for the continuous use of antimicrobials for the treatment diarrhea (Dupont H. L., C. D. Ericsson, J. J. Mathewson, M. W. Dupont, Z. D. Jiang, A. Mosavi and F. J. de la Cabaña, 1998. Rifaximin: a nonabsorved antimicrobial in the therapy of traveler's diarrhea. Digestion 59: 708-714). The extensive use of antibiotics can also lead to overgrowth syndromes, and Candida vaginitis can occur. The overgrowth of Clostridium difficile due to a less competitive environment in the gastrointestinal tract can also result in diarrhea.

The microorganisms responsible for gastrointestinal problems include bacteria, fungi, and viruses present in food and water.

Treatment and/or prevention of these food borne infections have been in a number of ways. In addition to non-pharmaceutical treatments, such as hydration, pharmaceutical treatments can be used.

Non-pharmaceutical methods of prevention include good hygiene (e.g., hand washing and hygiene in food preparation and processing), healthy diet, and low stress.

Pharmaceutical treatments can include various antibiotics or other anti-infectives.

Most antibiotics have the following side effects (although specific antibiotics may have other side effects or fewer of the standard ones).

1) The most common side effect for nearly all antibiotics is gastrointestinal distress.

2) Antibiotics double the risk for vaginal infections in women.

3) Allergic reactions can also occur with all antibiotics but are most common with medications derived from penicillin or sulfa. These reactions can range from mild skin rashes to rare but severe, even life-threatening anaphylactic shock.

4) Certain drugs, including some over-the-counter medications, interact with antibiotics.

Of great concern is the emergence of common bacteria strains that are now resistant to many standard antibiotics. Although new powerful antibiotics continue to designed, they are expensive and are also prone to resistance eventually.

These previous compositions and methods have drawbacks which are discussed above. These include for example, resistance to antibiotics, allergic reactions, and various side effects.

Terpenes are widespread in nature, mainly in plants as constituents of essential oils. Their building block is the hydrocarbon isoprene (C₅H₈)ₙ. Terpenes have been found to be effective and nontoxic dietary anti-tumor agents which act through a variety of mechanisms of action (Crowell, P. L. and M. N. Gould, 1994. Chemoprevention and therapy of cancer by d-limonene. Crit. Rev. Oncog. 5(1): 1-22; Crowell, P. L., S. Ayoubi and Y. D. Burke, 1996. Antitumorigenic effects of limonene and perillyl alcohol against pancreatic and breast cancer. Adv. Exp. Med. Biol. 401: 131-136). Terpenes, i.e., geraniol, tocotrienol, perillyl alcohol, b-ionone, and d-limonene, suppress hepatic HMG-COA reductase activity, a rate limiting step in cholesterol synthesis, and modestly lower cholesterol levels in animals (Elson, C. E. and S. G. Yu, 1994. The chemoprevention of cancer by mevalonate-derived constituents of fruits and vegetables. J. Nutr. 124: 607-614). D-limonene and geraniol reduced mammary tumors (Elegbede, J. A., C. E. Elson, A. Qureshi, M. A. Tanner and M. N. Gould, 1984. Inhibition of DMBA-induced mammary cancer by monoterpene d-limonene. Carcinogenesis 5(5): 661-664; Elegbede, J. A., C. E. Elson, A. Qureshi, M. A. Tanner and M. N. Gould, 1986. Regression of rat primary mammary tumors following dietary d-limonene. J. Natl. Cancer Inst. 76(2): 323-325; Karlson, J., A. K. Borg, R. Unelius, M. C. Shoshan, N. Wilking, U. Ringborg and S. Linder, 1996. Inhibition of tumor cell growth by monoterpenes in vitro: evidence of a Ras-independent mechanism of action. Anticancer Drugs 7(4): 422-429) and suppressed the growth of transplanted tumors (Yu, S. G., P. J. Anderson and C. E. Elson, 1995. The efficacy of B-ionone in the chemoprevention of rat mammary carcinogenesis. J. Agri. Food Chem. 43: 2144-2147).

Terpenes have also been found to inhibit the in vitro growth of bacteria and fungi (Chaumont J. P. and D. Leger, 1992. Campaign against allergic moulds in dwellings. Inhibitor properties of essential oil geranium “Bourbon”, citronellol, geraniol and citral. Ann Pharm Fr 50(3): 156-166; Moleyar, V. and P. Narasimham, 1992. Antibacterial activity of essential oil components. Int. J. Food Microbiol. 16(4): 337-342; and Pattnaik, S., V. R. Subramanyan, M. Bapaji and C. R. Kole, 1997. Antibacterial and antifungal activity of aromatic constituents of essential oils. Microbios. 89(358): 39-46) and some internal and external parasites (Hooser, S. B., V. R. Beasly and J. J. Everitt, 1986. Effects of an insecticidal dip containing d-limonene in the cat. J. Am. Vet. Med. Assoc. 189(8): 905-908). Geraniol was found to inhibit growth of Candida albicansand Saccharomyces cerevisiae strains by enhancing the rate of potassium leakage and disrupting membrane fluidity (Bard, M., M. R. Albert, N.Gupta, C. J. Guuynn and W. Stillwell, 1988. Geraniol interferes with membrane functions in strains of Candida and Saccharomyces. Lipids 23(6): 534-538). B-ionone has antifungal activity which was determined by inhibition of spore germination, and growth inhibition in agar (Mikhlin, E. D., V. P. Radina, A. A. Dmitrossky, L. P. Blinkova and L. G. Button, 1983. Antifungal and antimicrobial activity of some derivatives of beta-ionone and vitamin A. Prikl. Biokhim. Mikrobiol. 19: 795-803; Salt, S. D., S. Tuzun and J. Kuc, 1986. Effects of B-ionone and abscisic acid on the growth of tobacco and resistance to blue mold. Mimicry the effects of stem infection by Peronospora tabacina. Adam. Physiol. Molec. Plant Path. 28: 287-297). Teprenone (geranylgeranylacetone) has an antibacterial effect on H. pylori (Ishii, E., 1993. Antibacterial activity of teprenone, a non water-soluble antiulcer agent, against Helicobacter pylori. Int. J. Med. Microbiol. Virol. Parasitol. Infect. Dis. 280(1-2): 239-243). Rosanol, a commercial product with 1% rose oil, has been shown to inhibit the growth of several bacteria (Pseudomonas, Staphylococus, E. coli, and H. pylori). Geraniol is the active component (75%) of rose oil. Rose oil and geraniol at a concentration of 2 mg/L inhibited the growth of H. pylori in vitro. Some extracts from herbal medicines have been shown to have an inhibitory effect in H. pylori, the most effective being decursinol angelate, decursin, magnolol, berberine, cinnamic acid, decursinol, and gallic acid (Bae, E. A., M. J. Han, N. J. Kim and D. H. Kim, 1998. Anti-Helicobacter pylori activity of herbal medicines. Biol. Pharm. Bull. 21(9) 990-992). Extracts from cashew apple, anacardic acid, and (E)-2-hexenal have shown bactericidal effect against H. pylori.

Solutions of 11 different terpenes were effective in inhibiting the growth of pathogenic bacteria in in vitro tests; levels ranging between 100 ppm and 1000 ppm were effective. The terpenes were diluted in water with 1% polysorbate 20 (Kim, J., M. Marshall, and C. Wei, 1995. Antibacterial activity of some essential oil components against five foodborne pathogens. J. Agric. Food Chem. 43: 2839-2845). Diterpenes, i.e., trichorabdal A (from R. Trichocarpa), has shown a very strong antibacterial effect against H. pylori (Kadota, et al., 1997).

There may be different modes of action of terpenes against microorganisms; they could (1) interfere with the phospholipid bilayer of the cell membrane, (2) impair a variety of enzyme systems (HMG-reductase), and (3) destroy or inactivate genetic material.

For the above reasons, and others, the present invention provides additional methods for preserving food products and thus preventing gastrointestinal infections that avoid the drawbacks of previous methods.

### SUMMARY OF THE INVENTION

In accordance with the purpose(s) of this invention, as embodied and broadly described herein, this invention relates to preservation of food and prevention of infections, especially GI infections.

Disclosed is a food preservative comprising an effective amount of at least one effective terpene. The present invention provides a composition for preserving food preventing an infection, especially a GI infection, in a subject comprising an effective amount of at least one effective terpene. The composition can be a solution, especially a true solution. The composition can further comprise a carrier, e.g., water. The composition can further comprise a surfactant and water.

The composition may be a solution of terpene and water.

The composition of invention can comprise a mixture of different terpenes or a terpene-liposome (or other vehicle) combination.

The terpene of the composition can comprise, for example, citral, pinene, nerol, b-ionone, geraniol, carvacrol, eugenol, carvone, terpeniol, anethole, camphor, menthol, limonene, nerolidol, famesol, phytol, carotene (vitamin A₁), squalene, thymol, tocotrienol, perillyl alcohol, bomeol, myrcene, simene, carene, terpenene, linalool, or mixtures thereof.

The composition is effective against various infective agents including bacteria, viruses, mycoplasmas, and/or fungi.

A method for preserving food is disclosed. The method comprises applying an effective amount of at least one effective terpene to unspoiled food.

A composition for preventing a gastrointestinal infection in a subject comprising a true solution comprising an effective amount of at least one effective terpene and water is also disclosed.

The application of the method can be by spraying on unspoiled food a solution containing a single bioactive terpene, a bioactive terpene mixture, or a liposome-terpene(s) composition with or without a surfactant.

The methods are practiced using the compositions of the present invention.

The composition can be made by mixing an effective amount of an effective terpene and water. The mixing can be done at a solution-forming shear until formation of a true solution of the terpene and water, the solution-forming shear may be by high shear or high pressure blending or agitation.

The invention includes a method for making a terpene-containing composition effective for preventing and/or treating infections comprising mixing a composition comprising a terpene and water at a solution-forming shear until a true solution of the terpene is formed.

The invention is also a method for making a terpene-containing composition effective for preventing and/or treating infections comprising adding terpene to water, and mixing the terpene and water under solution-forming shear conditions until a true solution of terpene and water forms.

The invention provides preservation of all types of food utilized for human or animal consumption by the addition of food grade terpenes with biocidal activity.

A method comprises addition, e.g., spraying or dipping food, of food grade terpenes.

Additional advantages will be set forth in part in the description which follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### DESCRIPTION OF THE PREFERRED EMBODIMENTS

Before the present compounds, compositions, articles, devices, and/or methods are disclosed and described, it is to be understood that this invention is not limited to specific synthetic methods. It is also to be understood that the terminology used herein is for the purpose of describing particular aspects only and is not intended to be limiting.

In this specification and in the claims which follow, reference will be made to a number of terms which shall be defined to have the following meanings:

Definitions

It must be noted that, as used in the specification and the appended claims, the singular forms “a,” “an,” and “the” include plural referents unless the context clearly dictates otherwise. Thus, for example, reference to “an aerosol” includes mixtures of aerosols, reference to “a terpene” includes mixtures of two or more such terpenes, and the like.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

References in the specification and concluding claims to parts by volume, of a particular element or component in a composition or article, denotes the volume relationship between the element or component and any other elements or components in the composition or article for which a part by volume is expressed. Thus, in a composition containing 2 parts by volume of component X and 5 parts by volume component Y, X and Y are present at a volume ratio of 2:5, and are present in such ratio regardless of whether additional components are contained in the composition.

A volume percent of a component, unless specifically stated to the contrary, is based on the total volume of the formulation or composition in which the component is included.

“Optional” or “optionally” means that the subsequently described event or circumstance may or may not occur, and that the description includes instances where said event or circumstance occurs and instances where it does not. For example, the phrase “optionally surfactant” means that the surfactant may or may not be added and that the description includes both with a surfactant and without a surfactant where there is a choice.

By the term “effective amount” of a compound or property as provided herein is meant such amount as is capable of performing the function of the compound or property for which an effective amount is expressed, such as a non-toxic but sufficient amount of the compound to provide the desired function, i.e., preservation. As will be pointed out below, the exact amount required will vary depending on the infective agent and conditions of the application, concentration of infective agent, the particular composition used, its mode of application, and the like. Thus, it is not possible to specify an exact “effective amount.” However, an appropriate effective amount may be determined by one of ordinary skill in the art using only routine experimentation.

By the term “effective terpene” is meant a terpene which is effective against the particular infective agent of interest.

As used throughout, by a “subject” is meant an individual. Thus, the “subject” can include domesticated animals (e.g., cats, dogs, etc.), livestock (e.g., cattle, horses, poultry, pigs, sheep, goats, etc.), and laboratory animals (e.g., mouse, rabbit, rat, guinea pig, etc.). In one aspect, the subject is a mammal, such as a primate or a human.

By the term “true solution” is meant a solution (essentially homogeneous mixture of a solute and a solvent) in contrast to an emulsion or suspension. A visual test for determination of a true solution is a clear resulting liquid. If the mixture remains cloudy, or otherwise not clear, it is assumed that the mixture formed is not a true solution but instead a mixture such as an emulsion or suspension.

By the term “food” is meant all foodstuffs including beverages.

Food preservation is performed by many methods. All methods may not be desirable or available in all circumstances. Preservation is performed to avoid occurrence of GI infections, among other reasons.

Digestive tract infections not only are an uncomfortable illness for humans, but in some cases can cause death in children, elderly, and immune-compromised people. The preferred treatment of the disease is antibiotics. The animal industry use of antibiotics has created the development of antibiotic-resistant bacteria. The increased antibiotic resistance has been the main reason to seek new antimicrobial alternatives. The European Community has banned the use of 5 antibiotics and in the Unites States, the FDA is banning the use of fluoroquinolone in animals due to the development of Campylobacter resistant to this antibiotic.

Terpenes, which are GRAS (Generally Recognized As Safe), have been found to inhibit the growth of cancerous cells, decrease tumor size, decrease cholesterol levels and have a biocidal effect on microorganisms in vitro. No prior reference has suggested the use of a terpene, terpene mixture, or liposome-terpene(s) combination for the prevention of gastrointestinal infections, e.g., traveler's diarrhea.

During processing of animals for human consumption, there are several areas where meat can become contaminated, during scalding, chilling, and especially during packaging. There are approved methods or additives for microbial control during chilling, but they are not allowed during packaging, although some will leach and provide a biocidal effect. We have found that by dipping or spraying carcasses with a solution containing terpenes, we can reduce the microbial load and the biocidal effect will remain when meat is packaged.

An aspect of this invention is that the terpene formulation can be tailored to obtain biocidal effect over a single type microorganism or to eliminate all types of microorganisms.

Another aspect of the present invention is that the same method can be used for the prevention of infections resulting from the intake of contaminated water and juices. The present invention can be added to water of unknown quality, as is the case of water in underdeveloped countries. The water in these countries sometimes has higher levels of Coliforms and other pathogenic organisms. The use of this invention can eliminate this problem. This invention can also be used in the juice and cider industry. There have been cases digestive infection resulting from the consumption of unpasteurized apple cider. The problem with apple cider is that it loses its flavor with heat sterilization. The use of this invention will allow the processor first to spray terpenes on apples to eliminate surface microorganisms and later add terpenes during bottling to extend shelf life and eliminate microorganism that have survived processing. This method can be used in a variety of drinks.

Another aspect of this invention is that can also be applied to machinery and hardware used in the processing of food. Biofilms are the source of much of the free-floating bacteria in drinking water and machinery, especially in pipes. Biofilms are a group of bacteria that have colonized the surface of pipes and machinery. Once bacteria colonize, they start forming a glycocalyx matrix that holds water, making a film of gelatinous and slippery consistency. Biofilms resist disinfection because the gel-like matrix provides a barrier. Terpenes are natural solvents, and that solvency can be greatly increased with the use of surfactants in the biocidal product. This invention provides for the elimination of microorganisms in biofilms by selecting the appropriate terpene mixture and the appropriate surfactant.

It will be apparent for those skilled in the art that the aforementioned objects and other advantages may be further achieved by the practice of the present invention.

The present invention has the capacity of reducing the incidences of GI infections. The composition comprises terpenes, which can be naturally-occurring chemicals that are found in plants, which are generally recognized as safe (GRAS) by the FDA. An aspect of this invention is that due to the mechanism of action, such as basic interference with cholesterol, terpenes do not generate microbial resistance. There are antimicrobial products containing terpenes, basically in the form of essential oils, but we have found that not all components of the essential oils are biocides.

Another aspect of the present invention is that by varying the concentration of terpenes different specificity and biocidal effect can be achieved and that by combining two or more terpenes in the same solution a synergistic effect can be obtained. A further aspect of this invention is that the terpenes and surfactant used are generally recognized as safe (GRAS) by the FDA. An additional aspect of this invention is that we can tailor the formulation and obtain biocidal effect over a single type microorganism or change the formulation and eliminate all types of microorganisms.

Applying one of the formulations of the present invention in spray form onto food or machinery reduces the amount of microorganism responsible for infections. Several formulations can be obtained by utilizing biocidal terpenes without departing from the principle of the present inventions.

Many plant extracts have biocidal and antioxidant properties, but their essential oil composition cannot be modified. We have found that certain biocidal terpenes, when in combination, have a synergistic biocidal effect and by changing the terpenes in the composition we can control some microorganism but no others. This means that we can tailor the terpene mixture in order to eliminate a multitude of infective agents, e.g., gram positive bacteria, gram negative bacteria, yeast, fungi, and even other parasites, including internal worms and viruses.

We have observed that the terpenes used in this invention can be targeted to different microorganisms. We have been able to prove the effectiveness of the present invention against microorganisms that are of importance for humans and animals. Also, the effective terpene amount can vary depending on the organism we are interested in eliminating.

This invention can be modified in several ways by adding or deleting from the formulation the type of terpene and surfactant.

The present invention includes methods of making the compositions and methods of using the compositions.

Composition(s)

The compositions of the present invention comprise isoprenoids. More specifically, the compositions of the present invention comprise terpenoids. Even more specifically, the compositions of the present invention comprise terpenes. Terpenes are widespread in nature, mainly in plants as constituents of essential oils. Terpenes are unsaturated aliphatic cyclic hydrocarbons. Their building block is the hydrocarbon isoprene (C₅H₈)ₙ. A terpene is any of various unsaturated hydrocarbons, such as C₁₀H₁₆, found in essential oils, oleoresins, and balsams of plants, such as conifers. Some terpenes are alcohols (e.g., menthol from peppermint oil), aldehydes (e.g., citronellal), or ketones.

Terpenes have been found to be effective and nontoxic dietary antitumor agents, which act through a variety of mechanisms of action. Crowell, P. L. and M. N. Gould, 1994. Chemoprevention and Therapy of Cancer by D-limonene, Crit. Rev. Oncog. 5(1): 1-22; Crowell, P. L., S. Ayoubi and Y. D. Burke, 1996, Antitumorigenic Effects of Limonene and Perillyl Alcohol Against Pancreatic and Breast Cancer, Adv. Exp. Med. Biol. 401: 131-136. Terpenes, i.e., geraniol, tocotrienol, perillyl alcohol, b-ionone and d-limonene, suppress hepatic HMG-COA reductase activity, a rate limiting step in cholesterol synthesis, and modestly lower cholesterol levels in animals. Elson C. E. and S. G. Yu, 1994, The Chemoprevention of Cancer by Mevalonate-Derived Constituents of Fruits and Vegetables, J. Nutr. 124: 607-614. D-limonene and geraniol reduced mammary tumors (Elgebede, J. A., C. E. Elson, A. Qureshi, M. A. Tanner and M. N. Gould, 1984, Inhibition of DMBA-Induced Mammary Cancer by Monoterpene D-limonene, Carcinogensis 5(5): 661-664; Elgebede, J. A., C. E. Elson, A. Qureshi, M. A. Tanner and M. N. Gould, 1986, Regression of Rat Primary Mammary Tumors Following Dietary D-limonene, J. Nat'l Cancer Institute 76(2): 323-325; Karlson, J., A. K. Borg, R. Unelius, M. C. Shoshan, N. Wilking, U. Ringborg and S. Linder, 1996, Inhibition of Tumor Cell Growth By Monoterpenes In Vitro: Evidence of a Ras-Independent Mechanism of Action, Anticancer Drugs 7(4): 422-429) and suppressed the growth of transplanted tumors (Yu, S. G., P. J. Anderson and C. E. Elson, 1995, The Efficacy of B-ionone in the Chemoprevention of Rat Mammary Carcinogensis, J. Angri. Food Chem. 43: 2144-2147).

Terpenes have also been found to inhibit the in vitro growth of bacteria and fungi (Chaumont J. P. and D. Leger, 1992, Campaign Against Allergic Moulds in Dwellings, Inhibitor Properties of Essential Oil Geranium “Bourbon, ” Citronellol, Geraniol and Citral, Ann. Pharm. Fr 50(3): 156-166), and some internal and external parasites (Hooser, S. B., V. R. Beasly and J. J. Everitt, 1986, Effects of an Insecticidal Dip Containing D-limonene in the Cat, J. Am. Vet. Med. Assoc. 189(8): 905-908). Geraniol was found to inhibit growth of Candida albicans and Saccharomyces cerevisiae strains by enhancing the rate of potassium leakage and disrupting membrane fluidity (Bard, M., M. R. Albert, N. Gupta, C. J. Guuynn and W. Stillwell, 1988, Geraniol Interferes with Membrane Functions in Strains of Candida and Saccharomyces, Lipids 23(6): 534-538). B-ionone has antifungal activity which was determined by inhibition of spore germination and growth inhibition in agar (Mikhlin E. D., V. P. Radina, A. A. Dmitrossky, L. P. Blinkova, and L. G. Button, 1983, Antifungal and Antimicrobial Activity of Some Derivatives of Beta-Ionone and Vitamin A, Prikl Biokhim Mikrobiol, 19: 795-803; Salt, S. D., S. Tuzun and J. Kuc, 1986, Effects of B-ionone and Abscisic Acid on the Growth of Tobacco and Resistance to Blue Mold, Mimicry the Effects of Stem Infection by Peronospora Tabacina, Adam Physiol. Molec. Plant Path 28:287-297). Teprenone (geranylgeranylacetone) has an antibacterial effect on H. pylori (Ishii, E., 1993, Antibacterial Activity of Terprenone, a Non Water-Soluble Antiulcer Agent, Against Helicobacter Pylori, Int. J. Med. Microbiol. Virol. Parasitol. Infect. Dis. 280(1-2): 239-243). Solutions of 11 different terpenes were effective in inhibiting the growth of pathogenic bacteria in in vitro tests; levels ranging between 100 ppm and 1000 ppm were effective. The terpenes were diluted in water with 1% polysorbate 20 (Kim, J., M. Marshall and C. Wei, 1995, Antibacterial Activity of Some Essential Oil Components Against Five Foodborne Pathogens, J. Agric. Food Chem. 43: 2839-2845). Diterpenes, i.e., trichorabdal A (from R. Trichocarpa), have shown a very strong antibacterial effect against H. pylori (Kadota, S., P. Basnet, E. Ishii, T. Tamura and T. Namba, 1997, Antibacterial Activity of Trichorabdal A from Rabdosia Trichocarpa Against Helicobacter Pylori, Zentralbl. Bakteriol 287(1): 63-67).

Rosanol, a commercial product with 1% rose oil, has been shown to inhibit the growth of several bacteria (Pseudomona, Staphylococus, E. coli, and H. pylori). Geraniol is the active component (75%) of rose oil. Rose oil and geraniol at a concentration of 2 mg/L inhibited the growth of H. pylori in vitro. Some extracts from herbal medicines have been shown to have an inhibitory effect in H. pylori, the most effective being decursinol angelate, decursin, magnolol, berberine, cinnamic acid, decursinol, and gallic acid (Bae, E. A., M. J. Han, N. J. Kim, and D. H. Kim, 1998, Anti-Helicobacter Pylori Activity of Herbal Medicines, Biol., Pharm. Bull. 21(9) 990-992). Extracts from cashew apple, anacardic acid, and (E)-2-hexenal, have shown bactericidal effect against H. pylori. There may be different modes of action of terpenes against microorganism; they could (1) interfere with the phospholipid bilayer of the cell membrane, (2) impair a variety of enzyme systems (HMG-reductase), and (3) destroy or inactivate genetic material.

It is believed that due to the modes of action of terpenes being so basic, e.g., blocking of cholesterol, that infective agents will not be able to build a resistance to terpenes.

Terpenes, which are Generally Recognized as Safe (GRAS) have been found to inhibit the growth of cancerous cells, decrease tumor size, decrease cholesterol levels, and have a biocidal effect on microorganisms in vitro. Owawumni, G. O., 1989, Evaluation of the Antimicrobial Activity of Citral, Letters in Applied Microbiology 9(3): 105-108, showed that growth media with more than 0.01% citral reduced the concentration of E. coli, and at 0.08% there was a bactericidal effect. Barranx, A. M. Barsacq, G. Dufau, and J. P. Lauilhe, 1998, Disinfectant or Antiseptic Composition Comprising at Least One Terpene Alcohol and at Lease One Bactericidal Acidic Surfactant, and Use of Such a Mixture, U.S. Pat. No. 5,673,468, teach a terpene formulation, based on pine oil, used as a disinfectant or antiseptic cleaner. Koga, J. T. Yamauchi, M. Shimura, Y. Ogasawara, N. Ogasawara and J. Suzuki, 1998, Antifungal Terpene Compounds and Process for Producing the Same, U.S. Pat. No. 5,849,956, teach that a terpene found in rice has antifungal activity. Iyer, L. M., J. R. Scott, and D. F. Whitfield, 1999, Antimicrobial Compositions, U.S. Pat. No. 5,939,050, teach an oral hygiene antimicrobial product with a combination of 2 or 3 terpenes that showed a synergistic effect. Several U.S. patents (U.S. Pat. Nos. 5,547,677, 5,549,901, 5,618,840, 5,629,021, 5,662,957, 5,700,679, 5,730,989) teach that certain types of oil-in-water emulsions have antimicrobial, adjuvant, and delivery properties.

Terpenes are widespread in nature. Their building block is the hydrocarbon isoprene (C₅H₈)ₙ. Examples of terpenes include citral, pinene, nerol, b-ionone, geraniol, carvacrol, eugenol, carvone, terpeniol, anethole, camphor, menthol, limonene, nerolidol, farnesol, phytol, carotene (vitamin A₁), squalene, thymol, tocotrienol, perillyl alcohol, borneol, myrcene, simene, carene, terpenene, and linalool.

An effective terpene of the composition can comprise, for example, citral, pinene, nerol, b-ionone, geraniol, carvacrol, eugenol, carvone, terpeniol, anethole, camphor, menthol, limonene, nerolidol, farnesol, phytol, carotene (vitamin A₁), squalene, thymol, tocotrienol, perillyl alcohol, borneol, myrcene, simene, carene, terpenene, linalool, or mixtures thereof. More specifically, the terpene can comprise citral, carvone, eugenol, b-ionone, or mixtures thereof.

The composition can comprise an effective amount of the terpene. By the term “effective amount” of a composition as provided herein is meant a nontoxic but sufficient amount of the composition to provide the desired result. An appropriate effective amount can be determined by one of ordinary skill in the art using only routine experimentation.

The composition can comprise between about 100 ppm and about 2000 ppm of the terpene, specifically 100, 250, 500, or 1000 ppm.

A composition of the present invention comprises an effective amount of an effective terpene. An effective (i.e., preservative or anti-infective) amount of the effective terpene is the amount that produces a desired effect, i.e., preservation of food and/or prevention of an infection. This is the amount which will kill the infective agent. Less than a full kill may be effective. However, it is relatively easy to adjust the amount to achieve a full kill. If there were an instance where the amount for a full kill was very close to the toxic amount for a subject to ingest, an amount that achieves a stable population or stasis of the infective agent may be sufficient to prevent disease. An effective (i.e., preservative or anti-infective) terpene is one which produces the desired effect, i.e., preservation of food and/or prevention of infection, against the particular infective agent(s) with the potential to infect the subject(s).

The most effective terpenes can be the C₁₀H₁₆ terpenes. The more active terpenes for this invention can be the ones which contain oxygen. It is preferred for regulatory and safety reasons that at least food grade terpenes (as defined by the U.S. FDA) be used.

The composition can comprise a single terpene, more than one terpene, a liposome-terpene combination, or combinations thereof. Mixtures of terpenes can produce synergistic effects.

All classifications of natural or synthetic terpenes will work in this invention, e.g., monoterpenes, sesquiterpenes, diterpenes, triterpenes, and tetraterpenes. Examples of terpenes that can be used in the present invention are citral, pinene, nerol, b-ionone, geraniol, carvacrol, eugenol, carvone, terpeniol, anethole, camphor, menthol, limonene, nerolidol, famesol, phytol, carotene (vitamin A₁), squalene, thymol, tocotrienol, perillyl alcohol, borneol, myrcene, simene, carene, terpenene, and linalool. The list of exempted terpenes found in EPA regulation 40 C.F.R. Part 152 is incorporated herein by reference in its entirety. The terpenes may also be known by their extract or essential oil names, such as lemongrass oil (contains citral).

Citral, for example citral 95, is an oxygenated C₁₀H₁₆ terpene, C₁₀H₁₆O CAS No. 5392-40-5 3,7-dimethyl-2,6-octadien-1-al.

Terpenes are readily commercially available or can be produced by various methods known in the art, such as solvent extraction or steam extraction/distillation. Natural or synthetic terpenes are expected to be effective in the invention. The method of acquiring the terpene is not critical to the operation of the invention.

The liposome-terpene(s) combination comprises encapsulation of the terpene, attachment of the terpene to a liposome, or is a mixture of liposome and terpene. Alternatively, vehicles other than liposomes may be used, such as microcapsules or microspheres.

Liposomes are microscopic structures consisting of concentric lipid bilayers enclosing an aqueous space. Liposomes are classically prepared from phospholipids, which occur naturally in animal cell membranes, but several synthetic formulations are now commonly used. The lipid composition of the liposome can be varied to give liposomes different physical characteristics, e.g., size and stability. Liposomes can be prepared, for example, by the reverse-phase evaporation or dehydration-rehydration vesicle methods using a mixture of dipalmitoyl phosphatidyl choline, cholesterol, dipalmitoyl phosphatidyl glycerol, dipalmitoyl phosphatidyl ethanolamine, and/or other synthetic fatty acids and emulsifiers. When making liposomes first multilamellar vesicles are formed spontaneously when amphipathic lipids are hydrated in an aqueous medium. Unilamellar vesicles are often produced from multilamellar vesicles by the application of ultrasonic waves.

Multilamellar vesicles can be prepared by the procedure known as dehydration-rehydration. Briefly, e.g., egg phosphatidylcholine and cholesterol are mixed in chloroform, dried in a rotary evaporator, diluted with water, and sonificated to form unilamellar vesicles. The solution is freeze dried and rehydrated with the terpene solution in order to embed the terpene inside the liposome.

Another method to produce liposomes is by mixing together a lipid, an emulsifier, and a terpene. The emulsion can be obtained by using a Polytron® homogenizer with special flat rotor that creates an emulsion.

The lipid can comprise soybean oil, any commercial food grade or pharmaceutical oil; the emulsifier can comprise egg yolk lecithin, plant sterols or synthetic including polysorbate-80, polysorbate-20, polysorbate-40, polysorbate-60, polyglyceryl esters, polyglyceryl monooleate, decaglyceryl monocaprylate, propylene glycol dicaprilate and/or triglycerol monostearate. The lipid concentration in the oil phase can be about 75-95 vol %, and the emulsifier concentration from about 5-25 vol %. When preparing the emulsion a volumetric ratio of oil to water can vary from about 10-15 parts lipid to about 35-40 parts terpenes diluted in water at a concentration of about 0.5% to 50%. Once the emulsion is formed this is combined with a carrier in order to be used as a humectant, cream, or other suitable carrier for application. The emulsion concentration use for application can vary from, e.g., about 0.0055 to about 1.0% of the final product. Several modifications to the emulsion can be achieved by simply varying the concentration and type of terpenes used. This modifications can give us different products with different antimicrobial specificity.

By encapsulating terpenes within these emulsions the antimicrobial effect will be increased: (1) the liposome will disrupt the bacterial membrane and (2) the terpenes will be more effective in disrupting cytoplasmatic enzymes.

It is known to one of skill in the art how to produce a liposome or other encapsulating vehicle. For example, an oil-in-oil-in water composition of liposome-terpene may be used.

The composition can further comprise additional ingredients. For example, water (or alternatively, any bio-compatible or food grade or pharmaceutically acceptable dilutant or carrier), a surfactant, preservative, or stabilizer.

The surfactant can be non-ionic, cationic, or anionic. Examples of surfactant include polysorbate (Tween®) 20, polysorbate 80, polysorbate 40, polysorbate 60, polyglyceryl ester, polyglyceryl monooleate, decaglyceryl monocaprylate, propylene glycol dicaprilate, triglycerol monostearate, Span® 20, Span® 40, Span® 60, Span® 80, or mixtures thereof.

The composition can comprise 1 to 99% by volume terpenes and 0 to 99% by volume surfactant. More specifically the composition can comprise about 100 to about 2000 ppm terpenes and about 10% surfactant.

The concentration of terpene in the composition is an anti-infective or preserving amount. This amount can be from about an infective agent controlling level (e.g., about 10 or about 100 ppm) to about a level with side effects or possibly even a level toxic to a subject's cells (e.g., about 2000 ppm generally caused irritation in humans, though the level may be cell or subject specific). This amount can vary depending on the terpene(s) used, the form of terpene (e.g., liposome-terpene), the infective agent targeted, and other parameters that would be apparent to one of skill in the art. One of skill in the art would readily be able to determine an anti-infective amount for a given application based on the general knowledge in the art and the procedures in the Examples given below.

Specific compositions can include e.g.,

bacteria and fungi—1000 ppm terpenes in standard 0.9% saline with 50% 1-carvone, 30% eugenol, 10% purified eucalyptus oil, and 10% Tween® 80;

for mold—1000 ppm terpenes in water 100% citral or 95% citral and 5% Tween® 80; or

for mycoplasma—125 ppm or 250 ppm in PBS 95% b-ionone and 5% Tween(® 80.

Concentrations of terpene of 10, 15, 20, 25, 30, 35, 40, 45, 50, 55, 60, 65, 70, 75, 80, 90, 100, 110, 125, 130, 140, 150, 160, 175, 190, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1100, 1250, 1375, 1425, 1500, 1600, 1750, or 2000 ppm can be used as effective concentrations in the compositions and methods of the current invention.

Concentrations of any other ingredients or components can also be readily determined by one of skill in the art using methods known in the art and demonstrated below.

Terpenes have a relatively short life span of approximately 28 days once exposed to oxygen (e.g., air). Terpenes will decompose to CO₂ and water. This decomposition or break down of terpenes is an indication of the safety and environmental friendliness of the compositions and methods of the invention. The LD₅₀ in rats of citral is approximately 5 g/kg. This also is an indication of the relative safety of these compounds.

A stable suspension of citral can be formed up to about 2500 ppm. Citral can be made into a solution at up to about 1000 ppm. Of the terpenes tested, citral has been found to form a solution at the highest concentration level.

Citral will lyse human erythrocytes at approximately 1000 ppm. At sufficiently high levels of terpene, a terpene acts as a solvent and will lyse cell walls. Example 22 shows the levels that will lyse red blood cells.

A composition comprising a terpene, water, and a surfactant forms a suspension of the terpene in the water. Some terpenes may need a surfactant to form a relatively homogeneous mixture with water.

A composition comprising a “true” solution of a terpene is desired in order to minimize additional components which may cause undesired effects. A method for making a true solution comprising a terpene is described below.

The composition(s) of the present invention are effective against most infective agents. Examples of infective agents include fungi, viruses, bacteria, and mycoplasmas.

The terpenes, surfactants, or other components of the invention may be readily purchased or synthesized using techniques generally known to synthetic chemists. Methods for making specific and exemplary compositions of the present invention are described in detail in the Examples below.

In one aspect, the compositions described herein can be applied to unspoiled food.

The amounts of the compositions described herein are large enough to produce the desired effect in the method by which delivery occurs. The amount should not be so large as to cause adverse side effects.

The invention includes a method of making the composition of the present invention. A method of making a terpene-containing composition that is effective for preserving food and/or preventing GI infection comprises adding an effective amount of an effective terpene to a carrier solvent.

The terpenes and carriers are discussed above. The concentration at which each component is present is also discussed above. For example, 1000 ppm of citral can be added to water to form a true solution. As another example, 2000 ppm of citral can be added to water with a surfactant to form a stable suspension.

The method can further comprise adding a surfactant to the terepene-containing composition. Concentrations and types of surfactants are discussed above.

The method can further comprise mixing the terpene and carrier (e.g., water, saline, or buffer solution). The mixing is under sufficient shear until a “true” solution is formed. Mixing can be done via any of a number of high shear mixers or mixing methods. For example, adding terpene into a line containing water at a static mixer is expected to form a solution of the invention. With the more soluble terpenes, a true solution can be formed by agitating water and terpene by hand (e.g., in a flask). With lesser soluble terpenes, homogenizers, or blenders provide sufficient shear to form a true solution. With the least soluble terpenes, methods of adding very high shear are needed, or if enough shear cannot be created, can only be made into the desired mixture by addition of a surfactant.

Mixing the terpene and water with a solution-forming amount of shear instead of adding a surfactant will produce a true solution. A solution-forming amount of shear is that amount sufficient to create a true solution as evidenced by a final clear solution as opposed to a cloudy suspension or emulsion.

Citral is not normally miscible in water. Previously in the art, a surfactant has always been used to get such a terpene into solution in water. The present invention is able to form a solution of up to 1000 ppm in water by high shear mixing, and thus, overcome the necessity of a surfactant in all solutions. Of the terpenes tested, citral has been found to form a solution at the highest concentration level in water.

In a large-scale production, the terpene can be added in line with the water and the high shear mixing can be accomplished by a static inline mixer.

Any type of high shear mixer will work. For example, a static mixer, hand mixer, blender, or homogenizer will work.

Food borne infections are caused by a variety of organisms. For example,.these organisms include bacteria, viruses, mycoplasmas, or fungi. The present invention is effective against any of these classifications of infective agents, in particular, bacteria, mycoplasmas, and fungi.

The compositions and methods of the present invention are effective in preventing these infections in a great variety of subjects, including humans and animals.

The composition of this invention can be applied by a variety of means. For example, the composition can be applied by spraying food. Also, the food could be dipped, coated, and the like. Further, devices or articles touching the food during preparation or processing can be treated using the present compositions.

The life span/breakdown time of the terpenes, as indicated above, should be taken into account when formulating a treatment schedule for prevention or treatment according to the present invention.

### EXAMPLES

The following examples are put forth so as to provide those of ordinary skill in the art with a complete disclosure and description of how the compounds, compositions, articles, devices, and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.) but some errors and deviations should be accounted for. Unless indicated otherwise, parts are parts by volume, temperature is in ° C. or is at ambient temperature, and pressure is at or near atmospheric. There are numerous variations and combinations of the compositions and conditions for making or using them, e.g., component concentrations, desired solvents, solvent mixtures, temperatures, pressures, and other ranges and conditions that can be used to optimize the results obtained from the described compositions and methods. Only reasonable and routine experimentation will be required to optimize these.

### Example 1

### Preparation of the Terpene Mixture with Surfactant

The terpene, terpene mixture, or liposome-terpene(s) combination comprised a blend of generally recognized as safe (GRAS) terpenes with a GRAS surfactant. The volumetric ratio of terpenes was 1-99%, and the ratio of surfactant was 0-99% of the composition.

The terpenes, comprised of natural or synthetic terpenes, used were citral, b-ionone, eugenol, geraniol, carvone, terpeniol, carvacrol, anethole, or other terpenes with similar properties. The surfactant was polysorbate-80, Tween® 80, or other suitable GRAS surfactant. The terpenes were added to water.

### Example 2

### Preparation of a Terpene Solution Without Surfactant

Alternatively, the solution can be prepared without a surfactant by placing the terpene, e.g., citral, in water and mixing under solution forming shear conditions until the terpene is in solution.

The terpene-water solution was formulated without a surfactant. 100 ppm to 2000 ppm of natural or synthetic terpenes, such as citral, b-ionone, geraniol, carvone, terpeniol, or other terpenes with similar properties, were added to water and subjected to a high-shear blending action that forced the terpene(s) into a true solution. The terpene and water were blended in a household blender for 30 seconds. Alternatively, moderate agitation also prepared a solution of citral by shaking by hand for approximately 2-3 minutes.

The maximum level of terpene(s) that was solubilized varied with each terpene. Examples of these levels are as follows.

TABLE 1
Solution levels for various terpenes.

| Terpene | Level |
|---|---|
| Citral | 1000 ppm |
| Terpeniol | 500 ppm |
| b-ionone | 500 ppm |
| Geraniol | 500 ppm |
| Carvone | 500 ppm |

### Example 3

### Preparation of Liposomes Containing Terpenes

Any standard method for the preparation of liposomes can be followed with the knowledge that the lipids used are all food-grade or pharmaceutical-grade.

A fixed amount of lipid(s), emulsifier, and terpene(s) were used to prepare an emulsion. The emulsion was obtained by using a Polytron® homogenizer with a stainless-steel flat bottom rotor specific for liposome and emulsion production.

The lipids were soybean oil, any commercial food-grade, or pharmaceutical oil; the emulsifier was egg yolk lecithin, plant sterols, or synthetic including polysorbate-80, polysorbate-20, polysorbate-40, polysorbate-60, polyglyceryl esters, polyglyceryl monooleate, decaglyceryl monocaprylate, propylene glycol dicaprilate, and triglycerol monostearate.

A solution containing 75-95 vol % lipids (oil) and 5-25% emulsifier made up the oil phase. The aqueous phase was a terpene(s) diluted in water at a rate of 0.5 vol % to 50%.

To form the emulsion, a volumetric ratio of oil to water varying from 10-15 parts lipid (oil phase) to 35-40 parts terpene(s) (aqueous phase) was mixed.

The suspension containing the lipid, emulsifier and terpene(s) was emulsified with the Polytron® homogenizer until a complete milky solution was obtained.

### Example 4

### Preparation of Liposome

This Example illustrates the preparation of the terpene(s)-liposome combination by mixing 99 vol % of liposome and 1% of terpene mixture.

Several combinations of this formulation can be obtained by varying the amount of terpene and liposome from 1 vol % to 99%.

The liposomes are prepared as in Example 3 without the addition of terpenes in the formulation.

### Example 5

### Potency of Solution

Terpenes will break down in the presence of oxygen.

Citral, for example, is an aldehyde and will decay (oxygenate) over a period of days. A 500 ppm solution will lose half its potency in 2-3 weeks.

### Example 6

### In vitro Effectiveness of Terpenes Against Several Microorganisms

In vitro effectiveness of terpene compositions against various organisms was tested. The effectiveness of a terpene mixture solution comprising 10% by volume polysorbate-80, 10% b-ionone, 10% L-carvone, and 70% citral (lemon grass oil) against Escherichia coli, Salmonella typhimurium, Pasteurella mirabilis, Pseudomonas aeruginosa, Staphylococcus aureus, Candida albicans, and Aspergillius fumigatus was tested. The terpene mixture solution was prepared by adding terpenes to the surfactant. The terpene/surfactant was then added to water. The total volume was then stirred using a stir bar mixer.

Each organism, except A. fumigatus, was grown overnight at 35-37° C. in tryptose broth. A. fumigatus was grown for 48 hours. Each organism was adjusted to approximately 10⁵ organisms/ml with sterile saline. For the broth dilution test, terpene mixture was diluted in sterile tryptose broth to give the following dilutions: 1:500, 1:1000, 1:2000, 1:4000, 1:8000, 1:16000, 1:32000, 1:64000, and 1:128000. Each dilution was added to sterile tubes in 5 ml amounts. Three replicates of each series of dilutions were used for each test organism. One half ml of the test organism was added to each series and incubated at 35-37° C. for 18-24 hours. After incubation the tubes were observed for growth and plated onto blood agar. The tubes were incubated an additional 24 hours and observed again. The A. fumigatus test series was incubated for 72 hours. The minimum inhibitory concentration (MIC) for each test organism was determined as the highest dilution that completely inhibited the organism.

TABLE 2
Results of the inhibitory activity of different
dilutions of terpene composition.

| | Visual Assessment of | | | Growth After Subculture | | | Mean |
|---|---|---|---|---|---|---|---|
| | Growth* | | | to Agar Plates* | | | Inhibitory |
| Organism | 1 | 2 | 3 | 1 | 2 | 3 | Dilution |
| S. typhimurium | 500 | 500 | 500 | 500 | 500 | 500 | 500 |
| E. coli | 1000 | 1000 | 1000 | 1000 | 1000 | 1000 | 1000 |
| P. mirabilis | 1000 | 1000 | 1000 | 1000 | 1000 | 1000 | 1000 |
| P. aureginosa | NI** | NI | NI | NI | NI | NI | NI |
| S. aureus | 1000 | 1000 | 1000 | 1000 | 1000 | 1000 | 1000 |
| C. albicans | 1000 | 1000 | 1000 | 1000 | 1000 | 1000 | 1000 |
| A. fumigatus | 8000 | 16000 | 16000 | 8000 | 16000 | 16000 | 13300 |

*The results of the triplicate test with each organism as the reciprocal of the dilution that showed inhibition/killing.
**NI = not inhibited.

### Example 7

### In vitro effectiveness of different terpene formulations against Escherichia coli, Salmonella typhimurium, Pasteurella mirabilis, Pseudomonas aeruginosa, Staphylococcus aureus, Candida albicans, and Aspergillus fumigatus

This example shows the amount and types of terpenes from six different terpene formulations (Table 3) used for antimicrobial testing.

In the microbiological study, seven microorganisms including Escherichia coli, Salmonella typhimurium, Pasteurella mirabilis, Pseudomonas aeruginosa, Staphylococcus aureus, Candida albicans, and Aspergillus fumigatus were utilized. These microorganisms were selected in view that they are commonly present in infections and contaminate animal products utilized for human consumption. Each organism, except A. fumigatus, was grown overnight at 35-37° C. in tryptone broth. A. fumigatus was grown for 48 hours. Each organism was adjusted to approximately 10⁵ organisms/ml with sterile saline.

Each terpene formulation was diluted to 1:500, 1:1000, 1:2000, 1:4000, 1:8000, and 1:16000 in broth and/or saline.

Each terpene formulation dilution was added to sterile tubes in 5 ml amounts, and 5 ml of the test organism was added to each series and incubated for 1 hour. There were three replicates of each series of dilutions for each test organism.

After incubation, 0.5 ml of each tube was plated onto blood agar and incubated 18-24 hours at 35-37° C. The A. fumigatus test series was incubated for 72 hours at 25 C.

The minimum inhibitory concentration (MIC) for each test organism was determined as the highest dilution that completely inhibits the organism growth. The microbiological results are presented in Table 4.

TABLE 3
Terpene formulation used for antimicrobial testing.

| | Formulas | | | | | |
|---|---|---|---|---|---|---|
| | (vol %) | | | | | |
| Terpene/Ingredient | A | B | C | D | E | F |
| Citral | | 15 | | | 20 | 70 |
| Carvone | | | 55 | 55 | 35 | 10 |
| Eugenol | | | 35 | | 40 | 10 |
| b-ionone | 30 | 80 | 10 | 40 | | |
| Liposome | 70 | | | | | |
| Tween ® 80 | | 5 | 5 | 5 | 5 | 10 |

TABLE 4
Effect of terpene formulations on microorganism growth.
DILUTION AT WHICH MICROORGANISM
GROWTH WAS INHIBITED

| | Formula | | | | | |
|---|---|---|---|---|---|---|
| Organism | A | B | C | D | E | F |
| E. coli | NI | NI | NI | NI | 2000 | 1000 |
| P. aeruginosa | NI | NI | NI | NI | 2000 | NI |
| P. mirabilis | NI | NI | NI | NI | 1000 | 1000 |
| S. typhimurium | NI | NI | NI | NI | 2000 | 500 |
| S. aureus | NI | 4000 | 1000 | 4000 | 2000 | 1000 |
| C. albicans | NI | 1000 | 2000 | 2000 | 2000 | 1000 |
| A. fumigatus | I | NI | NI | NI | 500 | 13300 |

The results are expressed as the reciprocal of the dilution that showed biocidal effect.
NI = not inhibited

### Example 8

### In vitro effectiveness of terpenes against fungal microorganisms: Sclerotinta homeocarpa, Rhizoctonia solani, and Colletotrichum graminicola

Two terpene formulations were tested against Sclerotinta homeocarpa, Rhizoctonia solani, and Colletotrichum graminicola. Formula A contained 40 vol % eugenol, 35% 1-carvone, 20% citral, and 5% Tween® 80. Formula B contained 70 vol % citral, 10% b-ionone, 10% 1-carvone, and 10% Tween(® 80.

Potato dextrose agar media was amended with each terpene formulation to make a 5000 ppm final concentration of each.

For each pathogen, a 5 mm diameter agar plug containing fungal micelia was transferred to each of 5 plates for both terpene formulations and a control. All plates were parafilmed and incubated at 25° C. The diameter of fungal colony growth was measured (mm) and recorded. When the control plates were full, measurements were stopped. Colony area was calculated using πr², where r is the radius of the colony.

TABLE 5
Effect of terpenes on fungal growth (area = mm²).

| | S. homeocarpa | | R. solani | | C. graminicola | |
|---|---|---|---|---|---|---|
| Treatment | Day 1 | Day 2 | Day 1 | Day 2 | Day 2 | Day 7 |
| Formula A | 0 | 0 | 0 | 0 | 0 | 0 |
| Formula B | 0 | 0 | 0 | 0 | 0 | 0 |
| Control | 209.0 | 2023.2 | 162.3 | 1976.6 | 136.7 | 2023.2 |

### Example 9

### In vitro Effectiveness of Single or Combination of Terpenes Against E. coli

The objective of this example was to determine a terpene mixture that could have an optimal biocidal effect.

E. coli strain AW574 was grown in tryptone broth to an exponential growth phase (O.D. between 0.4 and 1.0 at 590 nm). One tenth of this growth was inoculated to 10 ml of tryptone broth followed by the addition of individual terpenes or as indicated on Table 8; then incubated for 24 hours at 35-37° C., and the O.D. determined in each tube. The concentration of terpenes was 1 or 2 μMol. Each treatment was repeated in triplicate. The results are expressed as percentage bacterial growth as compared to the control treatment.

It is observed that the combination of terpenes gives better biocidal effect than single terpenes, with geraniol and carvone appearing to be better than b-ionone.

TABLE 6
Effect of single terpene or their combination against
E. coli growth.

| μMol terpenes | | | % |
|---|---|---|---|
| b-ionone | Carvone | Geraniol | growth |
| 0 | 0 | 0 | 100.00 |
| 2 | 0 | 0 | 84.00 |
| 0 | 2 | 0 | 63.00 |
| 0 | 0 | 2 | 54.00 |
| 1 | 1 | 1 | 41.00 |
| 1 | 2 | 1 | 31.10 |
| 1 | 1 | 2 | 14.80 |
| 1 | 2 | 2 | 15.90 |
| 2 | 1 | 1 | 48.60 |
| 2 | 2 | 1 | 44.30 |
| 2 | 1 | 2 | 30.20 |
| 2 | 2 | 2 | 1.50 |

### Example 10

### Summary of Mold Studies

TABLE 7
Formulas tested.

| Terpene | | | | | | | |
|---|---|---|---|---|---|---|---|
| (vol %) | A | FW | B | C | D | E | F |
| Citral | 20 | 70 | 10 | 30 | 60 | 100 | 95 |
| l-Carvone | 35 | 10 | 50 | 30 | 30 | — | — |
| Eugenol | 40 | — | 30 | 30 | — | — | — |
| b-ionone | — | 10 | — | — | — | — | — |
| Tween ® 80 | 5 | 10 | 10 | 10 | 10 | — | 5 |

Study 1:

1. Mold spores, Penicillum sp., were mixed with 1000 ppm of terpene formulation as indicated in Table 7 and added to a Potato-Dextrose agar plate.

2. After 48 h incubation, the plates showed the following results:

F<E<C<FW<D<A<B<Control.

3. After 72 hours, the plates showed the following results:

F<E<C<FW<D<A<B<Control.

Formulas F and E performed better than the others.

Study 2:

1.Mold spores, Penicillum sp., were mixed with 1000 ppm of each terpene formulation, incubated for 1 hour, and then added to Potato-Dextrose agar plates.

2. After 48 h incubation, the plates showed the following results:

F<FW<E<D<C<B<A<Control.

Formulas F and E performed better than the others.

Study 3:

1.Mold spores, Penicillum sp., were mixed with 1000 ppm of each terpene formulation, incubated for 24 hours, and then added to Potato-Dextrose agar plates.

2. After 48 h incubation, the plates showed the following results:

Ti F<E<D<FW<C<A<B<Control

Formulas F and E performed better than the others.

Tests were repeated several times with the same results. Formulas E and F performed better that the others.

### Example 11

### Biofilm Formation and Testing

### (Destruction of Biofilm)

Procedure:

In 96-well polystyrene plates or PVC plates

1. Add 100 ml of bacterial culture in nutrient broth, culture has to be made fresh by adding 1-2 ml of 1×10⁶ cfu in 50-100 ml broth and incubating overnight (14-18 h) at 37° C.

2. Incubate overnight at 35-37° C. This will develop a biofilm.

3. Wash 4 times with water.

4. Add 100 ml of 1:1000 terpene solution.

5. Let incubate for 1 hour or more depending on test protocol.

6. Add 25 μl of 1% crystal violet. This is done to quantify the biofilm formation. Dye will coat bacteria attached to wells.

7. Incubate for 15 minutes.

8. Wash wells four times with water and blot dry.

9. Add 200 μl 95% ethanol, mix.

10. In a new plate, transfer 150 μl solution to clean wells.

11. Read at 590 nm.

12. Results are expressed as the difference between O.D. of control and the treated samples.

Study 1:

Four terpene formulations with two type of surfactants, a total of eight formulas (A, B, C and D with 10% Tween® 80, H, J, K and L have 10% Span® 20) were prepared. Formulas A-D are those used in Example 10 with 10% Tween® 80. H-L are Formulas A-D from Example 10 with 10% Span® 20.

TABLE 8
Formulas tested vs. control for reduction in biofilm achieved.

| Formula | O.D. test | O.D. control | % reduction |
|---|---|---|---|
| A | 0.098 | 0.210 | 53 |
| B | 0.187 | 0.220 | 15 |
| C | 0.220 | 0.229 | 4 |
| D | 0.295 | 0.230 | 0 |
| H | 0.223 | 0.230 | 3 |
| J | 0.273 | 0.194 | 0 |
| K | 0.233 | 0.194 | 0 |
| L | 0.153 | 0.194 | 0 |

Study 2:

Destruction of Biofilm by Terpenes

Five formulas with their results. The formulas correspond to those used in Example 10.

TABLE 9
Formulas tested vs. control for reduction in biofilm achieved

| Formula | O.D. test | O.D. control | % reduction |
|---|---|---|---|
| A | 0.299 | 0.459 | 35 |
| FW | 0.437 | 0.459 | 5 |
| B | 0.284 | 0.459 | 38 |
| C | 0.264 | 0.459 | 42 |
| D | 0.247 | 0.459 | 46 |

### Example 12

### Biofilm Formation and Testing

### (Prevention of Biofilm Formation)

Procedure:

In 96-well polystyrene plates or PVC plates

1. Add 50 ml of bacterial culture in nutrient broth, culture has to be made fresh by adding 1-2 ml of 1×10⁶ cfu in 50-100 ml broth and incubating overnight (14-18 h) at37° C.

2. Add 100 ml of 1:1000 terpene solution.

3. Incubate overnight at 35-37° C. This will develop a biofilm.

4. Wash 4 times with water.

5. Add 25 μl of 1% crystal violet. This is done to quantify the biofilm formation. Dye will coat bacteria attached to wells.

6. Incubate for 15 minutes.

7. Wash wells four times with water and blot dry.

8. Add 200 μl 95% ethanol, mix.

9. In a new plate, transfer 150 μl solution to clean wells.

10. Read at 590 nm.

11. Results are expressed as the difference between O.D. of control as compared to treated samples.

### Example 13

### Determination of Citral in Water Samples

Reagents: Schiff reagent is diluted 1:10 with distilled water.

1. In test tubes, add 1 ml of solution to be tested.

2. Add 0.1 ml of 1:10 Schiff reagent.

3. Incubate at room temperature for 10 minutes.

4. Reaction will turn from pink to blue, pink color is 0 ppm citral, reaction starts to turn blue above 100 ppm.

### Example 14

### In Vitro Effectiveness of Terpenes Against Mycoplasma pneumoniae

Terpene beta-ionone or L-carvone was first mixed well with Tween® 80 to have a final Tween®80 concentration of 5 vol %. This mixture was then used to make concentrations of 2500 ppm in sterile phosphate buffer saline (PBS) by blending the mixture in PBS for 40 seconds. This 2500 ppm solution was then diluted to 500 ppm, 250 ppm, and 125 ppm with PBS.

PBS containing 25 ppm Tween® 80 or PBS alone was used to treat cells suspension as controls.

A log phase (2-3-day old) culture of Mycoplasma pneumoniae was mixed with each of the above three concentrations of terpene at 1: 1 (volume) ratio (in this case, 1 mL of cell suspension was added to 1 mL of terpene).

The culture and terpene mixture was then incubated at 37° C. for 40 hours. After 40 hours of treatment, 10-fold serial dilution was performed to 10 (−10) by first taking 0.1 mL of the treated culture suspension was added into 0.9 mL of fresh SP4 (Whitcomb (1983); SP4 media is commercially available (Remel, Lenexa, Kans., USA)). All the tubes were then incubated at 37° C., and a color change of the medium was used for the indication of the cells that either were killed or survived from the treatment. Color change was from red to yellow because Mycoplasma pneumoniae produces acid during its growth.

Three days after the 10-fold dilution, the first tube of the following treatments has changed color from red to yellow indication no killing effects:

PBS, PBS containing 25 ppm Tween® 80, 62.5 ppm L-carvone, 125 ppm L-carvone, and 250 ppm L-carvone,

whereas those treated with 62.5 ppm, 125 ppm, and 250 ppm of beta-ionone did not change color at all indicating a killing effect of ionone on Mycoplasma pneumoniae. However, 6 days after the 10-fold dilution, the second and third tube of the PBS, PBS containing 25 ppm Tween® 80, 62.5 ppm L-carvone, 125 ppm L-carvone, and 250 ppm L-carvone changed color,

whereas only the first tube of 62.5 ppm beta-ionone changed color indicating that beta-ionone at 125 and 250 ppm may have completely killed all cells in 40 hours.

All the treatments were performed in duplicate.

### Example 15

### Biofilm Formation and Testing

### (Destruction of Biofilm)

TABLE 10
Terpene formulations.

| Terpene (%) | PL | PL-20 | B | FP | FP-20 | IB | IB-20 |
|---|---|---|---|---|---|---|---|
| Citral | 20 | 20 | 10 | 30 | 30 | 70 | 70 |
| L-carvone | 35 | 35 | 50 | 30 | 30 | 10 | 10 |
| Eugenol | 40 | 40 | 30 | 30 | 30 | — | — |
| B-ionone | — | — | — | — | — | 10 | 10 |
| Tween ® 80 | 5 | — | 10 | 10 | — | 10 | — |
| Span ® 20 | — | 5 | — | — | 10 | — | 10 |

Procedure:
In a 96-well PVC plate
1. Add 100 ml of bacterial culture in nutrient broth, culture has to be made fresh by adding 1-2 ml 1 × 10⁶ cfu in 50-100 ml broth and incubating overnight (14-18 h) at 37° C.
2. Incubate overnight at 35-37° C. This will develop a biofilm.
3. Wash 4 times with water.
4. Add 100 ml of 1:1000 terpene solution.
5. Let incubate for 1 hour.
6. Add 25 μl of 1% crystal violet. This is done to quantify the bioflim formation. Dye will coat bacteria attached to wells.
7. Incubate for 15 minutes.
8. Wash wells four times with water and blot dry.
9. Add 200 μl 95% ethanol, mix.
10. In a new plate, transfer 150 μl solution to clean wells.
11. Read at 590 nm.
12. Results are expressed as the difference between O.D. of control as compared to treated samples after subtracting background O.D.

TABLE 11
Results.

| Formula | O.D. Terpene | O.D. Control | % decrease in O.D. |
|---|---|---|---|
| PL | 0.205 | 0.311 | −34 |
| PL-20 | 0.372 | 0.524 | −30 |
| IB | 0.516 | 0.650 | −21 |
| IB-20 | 0.463 | 0.505 | −8 |
| FP | 0.419 | 0.557 | −25 |
| FP-20 | 0.311 | 0.441 | −25 |

### Example 16

### Biofilm Formation and Testing

### (Prevention of Biofilm Formation)

Procedure:

In a 96-well PVC plate

1. Take 50 ml of bacterial culture in nutrient broth, that has made fresh by adding 1-2 ml 1×10⁶ cfu in 50-100 ml broth and incubated overnight (14-18 h) at 37° C.

2. Mix the bacterial broth with 50 μl of 1:1000 terpene solution (as shown in Example 15).

3. Incubate overnight at 35-37° C. This will develop a biofilm.

4. Wash 4 times with water.

5. Add 50 μl of 1% crystal violet. This is done to quantify the biofilm formation. Dye will coat bacteria attached to wells.

6. Incubate for 15 minutes.

7. Wash wells four times with water and blot dry.

8. Add 200 μl 95% ethanol, mix.

9. In a new plate, transfer 150 μl solution to clean wells.

10. Read at 590 nm.

11. Results are expressed as the difference between O.D. of control as compared to treated samples after subtracting background O.D.

TABLE 12
Results.

| | | | % |
|---|---|---|---|
| Treatment | O.D. Terpene | O.D. Control | reduction |
| IB-20 | 0.059 | 0.278 | 100 |
| FP | 0.044 | 0.266 | 100 |
| B | 0.048 | 0.305 | 100 |
| PL | 0.038 | 0.196 | 98 |
| PL-20 | 0.041 | 0.192 | 96 |
| IB | 0.040 | 0.185 | 98 |

### Example 17

### Determination of Best Formula

Study 1:

Procedure: Nutrient agar containing 2.2×10⁸ E. coli was added to 0.9 ml Butterfield buffer and 1.0 ml of 1:1000 terpene mixture. This mixture was diluted 1:10 four times. 10 The following formulations (from Table 10) were used PL, PL-20, FP, FP-20, IB, IB-20, and control. After mixing (no incubation time), 0.1 ml of the solution was plated on crystal-violet neutral red bile glucose (VRBD) agar and incubated at 37° C. for 18-24 hours.

TABLE 13
Results.

| | | % reduction |
|---|---|---|
| Formula | cfu | from control |
| PL | 5.6 × 10⁵ | 99.50 |
| PL-20 | 1.5 × 10⁶ | 0 |
| FP | 2.9 × 10⁵ | 99.80 |
| FP-20 | 2.4 × 10⁶ | 99.80 |
| IB | 1.0 × 10⁶ | 0 |
| IB-20 | 6.7 × 10⁴ | 99.94 |
| Control | 1.2 × 10⁶ | 0 |

Study 2:

Procedure: Nutrient agar containing 2.2×10⁶ E. coli was added to 0.9 ml Butterfield buffer and 1.0 ml of 1:1000 terpene mixture. This mixture was diluted 1:10 four times. The following formulations were used PL, PL-20, FP, FP-20, IB, IB-20, and control. After mixing (no incubation time) 0.1 ml of the solution was plated on VRBD agar and incubated at 37° C. for 18-24 hours.

TABLE 14
Results.

| | | % reduction |
|---|---|---|
| Formula | cfu | from control |
| PL | 18 | 99.9 |
| PL-20 | 229 | 99.9 |
| FP | 167 | 99.9 |
| FP-20 | 1 | 99.9 |
| IB | 4.5 × 10² | 99.9 |
| IB-20 | 2.3 × 10³ | 99.9 |
| Control | TNC | 0 |

TNC = too numerous to count

Study 3:

Procedure: Nutrient agar containing 2.2×10⁶ E. coli was added to 0.9 ml Butterfield buffer and 1.0 ml of 1:1000 terpene mixture. This mixture was diluted 1:10 twice. The following formulations were used PL, PL-20, FP, FP-20, IB, IB-20, and control. After one hour incubation, 0.1 ml of the solution was plated on VRBD agar and incubated at 37° C. for 18-24 hours.

TABLE 15
Results.

| | | % reduction |
|---|---|---|
| Formula | cfu | from control |
| PL | TNC | 0 |
| PL-20 | TNC | 0 |
| FP | 8.1 × 10³ | 99.9 |
| B | TNC | 0 |
| IB | 6.6 × 10³ | 99.9 |
| IB-20 | TNC | 0 |
| Control | TNC | 0 |

Study 4:

Procedure: Nutrient agar 0.1 ml containing 2.2×10⁶ E. coli was added to 0.9 ml Butterfield buffer and 1.0 ml of 1:1000 terpene mixture. This mixture was diluted 1:10 four times. The following formulations were used PL, PL-20, FP, FP-20, IB, IB-20, and control. After mixing (no incubation time), 0.1 ml of the solution was plated on VRBD agar and incubated at 37° C. for 18-24 hours.

TABLE 16
Results.

| | | % reduction |
|---|---|---|
| Formula | cfu | from control |
| PL | 183 | 99.9 |
| PL-20 | 146 | 99.9 |
| FP | 23 | 99.9 |
| FP-20 | 603 | 99.9 |
| IB | 225 | 99.9 |
| IB-20 | 1.5 × 10⁶ | 50.00 |
| Control | 3.0 × 10⁶ | 0 |

### Example 18

### In vitro Effectiveness of Terpenes Against E. coli

This example demonstrates the effect of terpenes on the cell membrane fragility of E. coli, which is considered indicative of other pathogenic bacteria such as Salmonella and Listeria.

Lysis of the cell membrane was monitored by the determination of galactosidase activity. B-galactosidase is a well-characterized cytosolic enzyme in bacteria. This enzyme is inducible in the presence of isopropyl-1-thiogalactosidase (IPTG) and assayed calorimetrically with substrate o-nitro-phenyl-B-D-galactoside (ONPG). ONPG is cleaved to release o-nitrophenol which has a peak absorbance at 420 nm.

Since intact E. coli is impermeable to both ONPG and the enzyme, the cells have to be lysed prior to enzymatic assay. Therefore, the ability of terpenes to lyse E. coli can be measured with this enzymatic assay and compared to known lysing agents.

The procedure used was as follows: E. coli strains AW574 or AW405 were cultured overnight in 10 ml tryptone broth with 1 nM IPTG at 35° C. Cells were allowed to grow after an absorbance equal to 0.9 was reached.

Cells were harvested, washed with phosphate buffer and resuspended to an absorbance equal to 0.5.

0.1 ml of the bacteria culture was added to 0.9 ml of buffer, warmed to 30° C., and then 80 μl of terpenes (85% terpenes and 15% polysorbate-80), 80 μl water (background), or 40 μl chloroform plus 40 μl 1% SDS in water (positive control) were added.

After the addition of the lysing agents the tubes were mixed for 10 seconds, and 0.2 ml of ONPG (4 mg/ml water) was added, then incubated for 5 minutes. The 5 enzyme activity was stopped with 0.5 ml of 1 M sodium carbonate. After being centrifuged for 3 minutes at 1,500×g, supernatant was transferred to cuvettes and read at 420 nm.

The relative degree of lysis caused by terpenes was calculated as follows: 100× (O.D. terpenes—O.D. water)/(O.D. chloroform—O.D. water).

This shows that dosages can be manipulated to either lyse the cell outright, or in the case of lower dosages, stop bacterial growth without lysis of the cell membrane. The advantage of this controllable result is the ability to prevent lysis and the resultant release of endotoxins where contraindicated.

TABLE 17
Lysis of E. coli by terpenes.

| Terpenes (μM) | | Relative lysis % |
|---|---|---|
| Carvone | 404,000 | NM* |
| | 40,400 | 54 |
| | 4,040 | 22 |
| | 404 | 3.2 |
| Geraniol | 363,000 | NM |
| | 36,300 | 96 |
| | 3,630 | 98 |
| | 363 | 34 |
| | 36.3 | 4 |
| | 3.63 | 2.4 |
| b-Ionone | 308,000 | NM |
| | 30,800 | NM |
| | 3,080 | NM |
| | 308 | 52 |
| | 30.8 | 44 |
| | 3.08 | 23 |
| | 0.308 | 4.78 |
| | 0.0308 | 1.3 |

| | |
|---|---|
| 80 μl Polysorbate-80 | 3.2 |
| 80 μl Polysorbate-80 + SDS + | 100 |
| Chloroform | |
| SDS + Chloroform | 100* |

*Lysis due to chloroform and SDS combination was considered to be 100%.
*NM = not measurable due to formation of turbid colloidal solution.

### Example 19

### In Vitro Effectiveness of Single or Combination of Terpenes Against E. coli

The objective of this example was to determine an optimum terpene mixture, which could have a greater biocidal effect.

E. coli strain AW574 was grown in tryptone broth to an exponential growth phase (O.D. between 0.4 and 1.0 at 590 nm).

One tenth of this growth was inoculated to 10 ml of tryptone broth followed by the addition of individual terpenes or as indicated on Table 18; then incubated for 24 hours at 35-37° C. and the O.D. determined in each tube.

The concentration of terpenes was 1 or 2 μMol. Each treatment was repeated in triplicate.

The results are expressed as percentage bacterial growth as compared to the control treatment.

It is observed that the combination of terpenes gives better biocidal effect than single terpenes, with geraniol and carvone better than b-ionone.

TABLE 18
Effect of single terpene or their combination against E. coli growth.

| μMol terpenes | | | % |
|---|---|---|---|
| B-ionone | Carvone | Geraniol | growth |
| 0 | 0 | 0 | 100.00 |
| 2 | 0 | 0 | 84.00 |
| 0 | 2 | 0 | 63.00 |
| 0 | 0 | 2 | 54.00 |
| 1 | 1 | 1 | 41.00 |
| 1 | 2 | 1 | 31.10 |
| 1 | 1 | 2 | 14.80 |
| 1 | 2 | 2 | 15.90 |
| 2 | 1 | 1 | 48.60 |
| 2 | 2 | 1 | 44.30 |
| 2 | 1 | 2 | 30.20 |
| 2 | 2 | 2 | 1.50 |

### Example 20

### In Vitro Effectiveness of Terpenes Against Escherichia coli Over Time

This example demonstrates the effectiveness of the terpene mixture at several concentrations against Escherichia coli and cultured over time.

Terpene dilutions (1:500, 1:1000, 1:2000, 1:4000, 1:8000, and 1:16,000) were prepared in brain heart infusion (BHI) broth and in saline. These were prepared in 25 ml amounts.

E. coli was grown overnight in BHI broth and diluted to a MacFarland 0.5 concentration in saline. This solution was diluted 1:100 to be used to inoculate (0.5 ml) each terpene dilution tube.

The series that contained the terpene dilution in BHI was tested at 30 min., 90 min., 150 min., and 450 min. Each tube was mixed and serially diluted in saline. 0.5 milliliters of each dilution was spread plated onto MacConkey (MAC) agar plates. Also, 3 drops of the undiluted and the 1:100 dilution was added into respective tubes of BHI broth. The tubes and plates were incubated overnight at 35° C.

The series that contained the terpene dilution in saline were tested at 60 min., 120 min., 180 min., and 480 min. Each tube was mixed and serially diluted in saline. 0.5 milliliters of each dilution was spread plated onto MacConkey (MAC) agar plates. Also, 3 drops of the undiluted and the 1:100 dilution were added into respective tubes of BHI broth. The tubes and plates were incubated overnight at 35° C.

TABLE 19
Subculture from the tubes containing various
dilutions of terpenes in broth.

| Time | Dilution | 1:500 | 1:1000 | 1:2000 | 1:4000 | 1:8000 | 1:16,000 |
|---|---|---|---|---|---|---|---|
| 30 | Un- | NG | + | + | + | + | + |
| min | diluted | | | | | | |
| | 1:100 | NG | + | + | + | + | + |
| 90 | Un- | NG | NG | + | + | + | + |
| min | diluted | | | | | | |
| | 1:100 | NG | NG | NG | + | + | + |
| 150 | Un- | NG | NG | + | + | + | + |
| min | diluted | | | | | | |
| | 1:100 | NG | NG | NG | + | + | + |
| 450 | Un- | NG | NG | + | + | + | + |
| min | diluted | | | | | | |
| | 1:100 | NG | NG | + | + | + | + |

NG = no growth, + = growth

TABLE 20
Subculture from the tubes containing various
dilutions of terpenes in saline.

| Time | Dilution | 1:500 | 1:1000 | 1:2000 | 1:4000 | 1:8000 | Control |
|---|---|---|---|---|---|---|---|
| 60 | Undiluted | NG | + | + | + | + | + |
| min | 1:100 | NG | NG | NG | + | + | + |
| 120 | Undiluted | NG | NG | NG | + | + | + |
| min | 1:100 | NG | NG | NG | NG | + | + |
| 180 | Undiluted | NG | NG | NG | + | + | + |
| min | 1:100 | NG | NG | NG | NG | + | + |
| 480 | Undiluted | NG | NG | NG | NG | + | + |
| min | 1:100 | NG | NG | NG | NG | NG | + |

NG = no growth, + = growth

TABLE 21
The quantitative results of the activity of various terpene
dilutions against E. coli (cfu).

| Media | Time | 1:500 | 1:1000 | 1:2000 | 1:4000 | 1:8000 | Control |
|---|---|---|---|---|---|---|---|
| Broth | 30 min | 0 | 0 | 660 | 3600 | 3600 | 4600 |
| | 90 min | 0 | 0 | 12 | 4600 | 5400 | 7600 |
| | 150 min | 0 | 0 | 10 | 8000 | 12,000 | 14,000 |
| | 450 min | 0 | 0 | 15,000 | 28 × | 23 × | 16 × |
| | | | | | 10³ | 10⁷ | 10⁸ |
| Saline | 60 min | 0 | 4 | 140 | 4000 | 2000 | 1300 |
| | 120 min | 0 | 0 | 0 | 90 | 3800 | 2600 |
| | 180 min | 0 | 0 | 0 | 2 | 2000 | 5000 |
| | 480 min | 0 | 0 | 0 | 0 | 104 | 8000 |

NG = no growth, + = growth

### Example 21

### Chicken Skin Test

This example shows the effectiveness of two terpene formulations (see Table 22) against Salmonella in a simulated poultry chiller scenario.

Two terpene formulations where diluted to four concentrations: 1:250, 1:500, 1:1000, and 1:2000.

Chicken skin inoculated with Salmonella typhimurium DT104 was dipped for 60 minutes at 4° C. in the respective terpene dilution (simulated poultry chiller).

Bacterial counts were obtained from viable populations in the solutions in which the inoculated skins were immersed, bacteria loosely attached to chicken skin, and bacteria firmly attached to the chicken skin.

TABLE 22
Recovery of Salmonella typhimurium DT104 from inoculated
broiler chicken skin.

| | Recovered Population (log 10 cfu/ml) | | | |
|---|---|---|---|---|
| | | | Loosely | Firmly |
| Terpene | | Suspended | Attached | Attached |
| Formulation | Dilution | Cells | Cells | Cells |
| Water | na | 3.45 | 4.14 | 3.34 |
| FW | 1:250 | 1.43 | 3.40 | 3.45 |
| FW | 1:500 | nd | 4.30 | 3.52 |
| FW | 1:1000 | 3.27 | 4.39 | 3.43 |
| FW | 1:2000 | 3.61 | 4.41 | 3.82 |
| Water | na | 3.38 | 4.20 | 3.33 |
| PL | 1:250 | <1 | 3.86 | 3.36 |
| PL | 1:500 | <1 | 4.03 | 3.51 |
| PL | 1:1000 | 2.84 | 4.27 | 3.70 |
| PL | 1:2000 | 3.45 | 4.28 | 3.63 |

na = not applicable
nd = not determined
FW formulation = citral 70 vol %, B-ionone 10%, L-carvone 10% and Tween ® 80 10%.
PL formulation = eugenol 40 vol %, L-carvone 35%, citral 20% and Tween ® 80 5%.

### Example 22

### Red Blood Cell Lysing Study

Protocol:

5 ml of PBS was added to terpene (100, 250, 500, 1000, and 2000 ppm). 0.050 ml of heparinized blood was then added to these mixtures. These mixtures were incubated for 20 minutes.

The samples were then centrifuged at 2000 rpm for 5 min and read at 540 nm.

Lysing of red cells in the terpene mixtures was compared to control, or to 0 ppm terpene.

Study 1: Chicken blood and B-ionone

TABLE 23
Destruction of red blood cells with b-ionone vs. control.

| | O.D. | |
|---|---|---|
| Concentration | B-ionone + | B-ionone |
| (ppm) | 10% Tween ® 80 | neat |
| 0 | 0.012 | 0.012 |
| 100 | 0.029 | 0.013 |
| 500 | 0.345 | 0.056 |
| 1000 | 0.576 | 0.038 |
| 2000 | 0.944 | 0.106 |

Lysing of the red cells occurred with terpene concentration of 500 ppm without Tween® and 100 ppm with Tween® 80.

Study 2: Human blood, b-ionone, citral, and 1-carvone

TABLE 24
Concentration at which erythrocytes lyse in the
presence of various terpenes.

| | Concentration at which | |
|---|---|---|
| | erythrocytes lyse | |
| | (ppm) | |
| Terpene | Neat | +10% Tween ® 80 |
| b-ionone | 2000 | 2000 |
| Citral | 1000 | 500 |
| l-Carvone | 250 | 250 |

This demonstrates the levels at which undesirable side effects can occur from terpenes if they enter the blood stream, for example, by ingestion and passing through the gastointestinal mucosa.

### Example 23

### Fruit Preservation

Study 1:

Reagents:

90 ml Butterfield buffer

19 g fresh grapes

10 ml veal infusion broth

0.1 ml 2.2×10⁸ E. coli

1:1000 terpenes solution (formulas from Table 10)

Procedure:

1. Make several of above preparations, depending on the number of treatments needed.

2. Incubate above mixture at 37° C. overnight, this will develop a biofilm on the grapes.

3. After incubation, wash grapes 5 times with water.

4. Rinse grapes in terpene solution (1:1000) for 5 minutes.

5. Wash grapes 5 times with water.

6. Add grapes to a container containing 100 ml Butterfield buffer.

7. Blend mixture with high speed blender.

8. Plates samples on VRBD (crystal-violet neutral red bile glucose) agar without dilution and with serial dilutions.

9. Incubate at 37° C. overnight.

10. Read plates.

TABLE 25
Results.

| | | % decrease over |
|---|---|---|
| Formula | cfu | control |
| Control | 2000 | 0 |
| PL | 700 | 65 |
| PL-20 | 1700 | 15 |
| FP | 80 | 96 |
| FP-20 | 1000 | 50 |

Study 2:

Performed as in Study 1.

TABLE 26
Results.

| | | % decrease over |
|---|---|---|
| Formula | cfu | control |
| Control | 2600 | 0 |
| PL | 1200 | 54 |

Throughout this application, various publications are referenced. The disclosures of these publications in their entireties are hereby incorporated by reference into this application in order to more fully describe the state of the art to which this invention pertains.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A composition for food preservation comprising an effective amount of at least one effective terpene.

2. The composition of claim 1 wherein the composition is a solution.

3. The composition of claim 1 further comprising water.

4. The composition of claim 1 further comprising a surfactant and water.

5. The composition of claim 4 wherein the surfactant is polysorbate 20, polysorbate 80, polysorbate 40, polysorbate 60, polyglyceryl ester, polyglyceryl monooleate, decaglyceryl monocaprylate, propylene glycol dicaprilate, triglycerol monostearate, Span® 20, Span® 40, Span® 60, Span® 80, or mixtures thereof.

6. The composition of claim 1 further comprising saline or a buffer solution.

7. The composition of claim 1 wherein the at least one terpene is a mixture of different terpenes.

8. The composition of claim 1 wherein the at least one terpene is a terpene-liposome combination.

9. The composition of claim 1 wherein the terpene comprises citral, pinene, nerol, b-ionone, geraniol, carvacrol, eugenol, carvone, terpeniol, anethole, camphor, menthol, limonene, nerolidol, famesol, phytol, carotene (vitamin A₁), squalene, thymol, tocotrienol, perillyl alcohol, borneol, myrcene, simene, carene, terpenene, linalool, or mixtures thereof.

10. The composition of claim 1 wherein the terpene is citral, carvone, b-ionone, eugenol, eucalyptus oil, or mixtures thereof.

11. The composition of claim 1 wherein composition comprises about 1 to 99% by volume terpenes and about 1 to 99% by volume surfactant.

12. The composition of claim 1 wherein the terpene comprises between about 10 ppm and about 2000 ppm.

13. The composition of claim 1 wherein the terpene comprises between about 100 ppm and about 2000 ppm.

14. The composition of claim 1 wherein the terpene comprises about 100 ppm.

15. The composition of claim 1 wherein the terpene comprises about 250 ppm.

16. The composition of claim 1 wherein the terpene comprises about 500 ppm.

17. The composition of claim 1 wherein the terpene comprises about 1000 ppm.

18. The composition of claim 1 wherein the terpene is 50% L-carvone, 30% eugenol, 10% purified eucalyptus oil and the effective amount is 1000 ppm, and wherein 10% is a surfactant.

19. The composition of claim 1 wherein the terpene is citral and the effective amount is 1000 ppm.

20. The composition of claim 19 wherein the composition further comprises 5% surfactant.

21. The composition of claim 1 wherein the terpene is b-ionone and the effective amount is 250 ppm and wherein 5% is a surfactant.

22. The composition of claim 1 wherein the terpene is effective against bacteria, mycoplasmas, and/or fungi.

23. The composition of claim 1 wherein the terpene is effective against bacteria.

24. The composition of claim 1 wherein the terpene is effective against mycoplasmas.

25. The composition of claim 1 wherein the terpene is effective against fungi.

26. The composition of claim 1 wherein the food is meat.

27. The composition of claim 1 wherein the food is fruit.

28. A composition for preserving food comprising a solution comprising an effective amount of at least one effective terpene and water.

29. A preservative comprising an effective amount of at least one effective terpene.

30. A method for preserving food comprising
applying a composition comprising an effective amount of an effective terpene to unspoiled food.

31. The method of claim 30 wherein the composition further comprises water.

32. The method of claim 30 wherein the composition further comprises a surfactant.

33. The method of claim 30 wherein the application is by spraying the unspoiled food with the composition.

34. The method of claim 30 wherein the application is by dipping the unspoiled food into the composition.

35. The method of claim 30 further comprising making a composition comprising
an effective amount of an effective terpene.

36. The method of claim 30 wherein the food is meat.

37. The method of claim 30 wherein the food is fruit.

38. The method of claim 35 wherein the making a composition comprises mixing an effective amount of an effective terpene and water.

39. The method of claim 38 wherein the mixing is done at a solution-forming shear until formation of a true solution of the terpene and water.

40. The method of claim 39 wherein the terpene mixed is into a true solution in water without a surfactant by high shear or high pressure blending or agitation.

41. The method of claim 40 wherein the solution-forming shear mixing is via a static mixer.

42. A method for preserving food comprising applying a composition comprising
an effective amount of an effective terpene and water to unspoiled food.

43. The method of claim 42 wherein the composition is a true solution.

44. A method for making a terpene-containing composition effective for preserving food comprising
mixing a composition comprising a terpene and water at a solution-forming shear until a true solution of the terpene is formed.

45. A method for making the composition of claim 1 comprising mixing a terpene with a carrier.

46. A method for using the composition of claim 1 comprising applying the composition of claim 1 to unspoiled food.

47. A method for preventing gastrointestinal infection from food carrying an infective agent comprising applying a composition comprising an effective amount of an effective terpene to unspoiled food.

48. A method for preventing gastrointestinal infection from food carrying an infective agent comprising applying a composition comprising an effective amount of an effective terpene to articles or devices for handling food.
